(19) United States
(12) Patent Application Publication
(10) Pub. No.: US 2025/0352296 A1
(43) Pub. Date: Nov. 20, 2025

(54) FLEXIBLE SURGICAL STENCILS AND RELATED METHODS

(71) Applicant: BAYLOR RESEARCH INSTITUTE D/B/A BAYLOR SCOTT & WHITE RESEARCH INSTITUTE, Dallas, TX (US)

(72) Inventor: Jack Leigh Eidson, III, Little River Academy, TX (US)

(21) Appl. No.: 19/209,087

(22) Filed: May 15, 2025

Related U.S. Application Data
(60) Provisional application No. 63/647,717, filed on May 15, 2024.

(51) Int. Cl. A61B 90/00; A61B 17/32; A61B 50/30

(52) U.S. Cl. CPC A61B 90/39; A61B 17/32; A61B 50/30; A61B 2017/320052; A61B 2090/3937; A61B 2090/3991

(57) ABSTRACT

A kit can comprise a flexible strap that extends between opposing first and second ends and has opposing upper and lower surfaces. The strap can have a plurality of openings that each extend through the strap from the upper surface to the lower surface, wherein the openings include a plurality of intermediate openings that are each disposed closer to the first end of the strap than is at least one other of the openings and closer to the second end of the strap than is at least one other of the openings. A length of the strap can be at least 20 times each of a thickness and a width of the strap. The strap can be sterile, and the kit can comprise a container that is sealed and contains the strap.

### CROSS-REFERENCE TO RELATED APPLICATIONS

This application claims the benefit of priority of U.S. Provisional Patent Application No. 63/647,717, filed May 15, 2024, which is hereby incorporated by reference in its entirety.

### BACKGROUND

### Field of the Invention

The present invention relates generally to surgical stencils to facilitate the planning and marking of incision guides for surgeries.

### Description of Related Art

During surgeries, incision guides are often marked on the skin of a patient to plan where a surgeon will makes the incisions required for surgery. In amputations, the incision typically surrounds the part of the body being removed. After removal of the part of the body, cuts of skin (also referred to as “flaps”) are joined to close the open wound.

The inventor has recognized challenges stemming from the current approach to marking and making incisions in such procedures. Currently, surgeons mark incision guides free-hand, using flimsy rulers that are not suited for the curvature of human body parts, or using suture string that serves as a poor guide for a surgical marker. These methods are susceptible to error, which can add time to the operation and can often yield misaligned cuts of skin of different lengths, which can cause wound complications to the patient after the cuts of skin are joined together.

### SUMMARY OF THE INVENTION

Thus, there is a need in the art for devices and techniques to accurately, effectively, and safely mark incision guides for surgical procedures. Various embodiments of the present kits and methods can address this need at least with one or more flexible straps that can be used as stencils to efficiently and accurately mark incision guides for surgical procedures. The flexible strap(s) can be configured in a way that helps ensure first and second segments of an incision guide marked using the flexible strap(s) have substantially similar lengths. As a result, cuts of skin along those segments of the incision guide can also have substantially the same length such that they can be better joined together to close an open wound after, for example, an amputation, which can reduce the risk of post-surgery complications.

To facilitate the accurate marking of such incision guides, the flexible strap(s) can each have a plurality of openings that each extend through the strap from the strap's upper surface to its lower surface. The openings can be sized such that fasteners—which can optionally be part of some of the present kits—can be inserted through two or more of the openings and into the patient's skin when the strap is disposed on the skin, effectively fixing the strap into place on the patient. Openings of the strap can have different positions along the strap's length to allow a surgeon to rely on the openings—and especially which of the openings have fasteners inserted therethrough—as a reference for the length of the segment of the incision guide being marked along the strap, as detailed further below.

In particular, to mark the first segment of the incision guide, one of the flexible strap(s) can be disposed on a first part of the skin and can be fastened thereto using fasteners extending through first and second ones of the strap's openings. With the strap fastened to the skin, in the portion of the strap extending between the first and second openings, one of the strap's lengthwise edges can define a desired incision path along which the first segment of the incision guide can be marked. In some embodiments, the flexible strap can then be removed from the first part of the skin, fastened to a second part of the skin with fasteners extending through the strap's first and second openings, and the second segment of the incision guide can be marked along one of the strap's lengthwise edges in the portion of the strap extending between the first and second openings. Using the same first and second openings for fastening the strap to the patient and employing the same portion of the strap extending between the first and second openings to mark the first and second segments of the incision guide can help efficiently ensure that the two segments have substantially the same length. To further facilitate the efficient, accurate marking of the first and second segments, first and second fastening points can be marked before marking the incision guide. The fastening points can designate where the strap will be attached to the patient when marking both the first and second segments of the incision guide, wherein to mark each segment, the strap is positioned such that the first opening—which can optionally be the opening closest to one of the ends of the strap—overlies one of the fastening points and the second opening overlies the other of the fastening points. This can facilitate the intersection between the first and second separately-drawn segments such that the incision guide circumscribes the body part.

In alternative embodiments, the first segment of the incision guide can be marked using the strap after the second segment of the incision guide is marked (e.g., using a conventional technique, such as free-hand). In such embodiments, the strap can be used to measure the length of the marked second segment, which can delineate which of the openings of the strap to use as the first and second openings for fastening the strap to the patient to mark the first segment. This can also facilitate the marking of segments of substantially the same length even with one of the segments drawn free-hand. Alternatively, two straps can be used, where a first strap can be used to mark the first segment and a second strap can be used to mark the second segment in the manner described above, with the straps either fastened to the patient at the same time or at different times. When using two straps, the lengthwise separation between the first and second openings of the first strap can be substantially the same as the lengthwise separation between the first and second openings of the second strap such that the segments drawn with the straps are of substantially the same length.

The first and second segments can delineate the guide along which the incision can be made. Because the second segment can have a length substantially similar to the length of the first segment, post-amputation, the opposing flaps of skin can have substantially the same length and allow for easy conjoining of the first and second parts of skin to finish the procedure.

The flexible strap(s) included in some of the present the kits can be sterile and disposed within a sealed container that optionally complies with ISO 11607-1:2019, which can render the strap(s) suitable for use in surgical procedures and mitigate the risk of infection.

Some of the present methods comprising marking an incision guide on skin of the patient. The incision guide, in some methods, circumscribes a part of a body of the patient. In some methods, the incision guide includes first and second vertices, a first segment that extends along a first part of the skin of the patient between the first and second vertices, and a second segment that extends along a second part of the skin of the patient between the first and second vertices. The second segment, in some methods, has a length, measured between the first and second vertices, that is substantially the same as a length, measured between the first and second vertices, of the first segment.

In some methods, marking the incision guide comprises disposing a flexible strap on a first part of the skin, and some of the present kits comprise a flexible strap. The strap, in some embodiments, extends between opposing first and second ends. In some embodiments, the strap has a plurality of openings that each extend through the strap from an upper surface of the strap to an opposing lower surface of the strap.

The openings, in some embodiments, include a plurality of intermediate openings, optionally ten or more intermediate openings, that are each disposed closer to the first end of the strap than is at least one other of the openings and closer to the second end of the strap than is at least one other of the openings. In some embodiments, for each of the openings of the strap, a distance between the opening and at least one other of the openings is less than or equal to 3 centimeters. Each of the openings, in some embodiments, has a maximum transverse dimension, measured in a direction that is perpendicular to the thickness of the strap, that is between 1 and 6 millimeters.

In some embodiments, a length of the strap, measured between the first and second ends, is at least 20 times each of a thickness and a width of the strap, the thickness measured between the upper and lower surfaces. The length of the strap, in some embodiments, is between 20 and 100 centimeters. In some embodiments, the thickness of the strap is between 1 and 10 millimeters. The width of the strap, in some embodiments, is between 1 and 10 millimeters. A cross-section of the strap that includes a plane that is perpendicular to the length of the strap, in some embodiments, is rectangular.

In some embodiments, the flexible strap comprises a polymeric material. A modulus of elasticity of the polymeric material, in some embodiments, is between 1.25 and 4 gigapascals.

Some kits comprise a plurality of fasteners. The fasteners, in some kits, each include a piercing body sized to extend through one of the openings of the flexible strap and configured to pierce skin of a patient. In some methods, marking the incision guide comprises fastening the strap to the first part of the skin of the patient at least by, for each of a first one of the openings of the strap and a second one of the openings of the strap that is disposed closer to the second end of the strap than is the first opening, inserting a piercing body of a fastener through the opening and into the skin of the patient. The first opening, in some methods, is one of the intermediate openings of the strap. In some methods, the second opening is one of the intermediate openings. In some methods, marking the incision guide comprises, while the strap is fastened to the first part of the skin of the patient, marking the first segment of the incision guide along one of opposing lengthwise edges of the strap that each extend between the upper and lower surfaces of the strap.

In some methods, marking the incision guide comprises, after marking the first segment of the incision guide, removing the strap from the first part of the skin of the patient. Marking the incision guide, in some methods, comprises, after removing the strap from the first part of the skin of the patient, disposing the strap on the second part of the skin of the patient. In some methods, marking the incision guide comprises, after removing the strap from the first part of the skin of the patient, fastening the strap to the second part of the skin of the patient at least by, for each of the first and second openings of the strap, inserting a piercing body of a fastener through the opening and into the skin of the patient. In some methods, marking the incision guide comprises, after removing the strap from the first part of the skin of the patient, while the strap is fastened to the second part of the skin of the patient, marking the second segment of the incision guide along one of the lengthwise edges of the strap.

Some methods comprise, before marking the incision guide, marking first and second fastening points on the skin of the patient. In some methods, the first opening of the strap overlies the first fastening point and the second opening of the strap overlies the second fastening point when the strap is fastened to the first part of the skin of the patient. In some methods, the first opening of the strap overlies one of the first and second fastening points and the second opening of the strap overlies the other of the first and second fastening points when the strap is fastened to the second part of the skin of the patient.

In some methods, a minimum distance between the first and second openings of the strap when the strap is fastened to the first part of the skin of the patient is substantially the same as a minimum distance between the first and second vertices of the incision guide.

Some methods comprise, after fastening the strap to the first part of the body of the patient, cutting the strap to remove a segment of the strap. The segment, in some methods, includes the first end of the strap or the second end of the strap. In some methods, the segment does not include the first and second openings of the strap.

Some methods comprise cutting the skin of the patient along the incision guide. Some methods comprise amputating the part of the body such that the patient has an open wound. Some methods comprise coupling the first part of the skin to the second part of the skin such that the first and second parts of the skin overlie the open wound.

The strap, in some kits, is sterile. In some kits, each of the fasteners is sterile. In some embodiments, a Sterility Assurance Level of the strap is less than or equal to 10⁻⁶. Some kits comprise a container is sealed and contains the strap. The container, in some kits, complies with ISO 11607-1:2019.

The term “coupled” is defined as connected, although not necessarily directly, and not necessarily mechanically. Two items that are “coupled” may be unitary with each other or may be connected to one another via one or more intermediate components or elements.

The terms “a” and “an” are defined as one or more unless this disclosure explicitly requires otherwise.

The term “substantially” is defined as largely, but not necessarily wholly, what is specified (and includes what is specified; e.g., substantially 90 degrees includes 90 degrees, and substantially parallel includes parallel), as understood by a person of ordinary skill in the art. In any disclosed embodiment, the terms “substantially” and “about” may be substituted with “within [a percentage] of” what is specified, where the percentage is 1, 5, or 10%.

The phrase “and/or” means and or or. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and containing”) are open-ended linking verbs. As a result, an apparatus that “comprises,” “has,” “includes,” or “contains” one or more elements possesses those one or more elements, but is not limited to possessing only those one or more elements. Likewise, a method that “comprises,” “has,” “includes,” or “contains” one or more steps possesses those one or more steps, but is not limited to possessing only those one or more steps.

Any embodiment of any of the apparatuses and methods can consist of or consist essentially of—rather than comprise/have/include/contain—any of the described elements, features, and/or steps. Thus, in any of the claims, the phrase “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate identical structures. Rather, the same reference numbers may be used to indicate similar features or features with similar functionalities, as may non-identical reference numbers.

FIG. 1 depicts a first embodiment of the present kits that includes a container that is sealed and contains a flexible strap and two fasteners usable to secure the strap to the patient. In FIG. 1, the strap is wound into a roll.

FIGS. 2A-2C depict a perspective view, top view, and side view, respectively, of the flexible strap of the kit of FIG. 1 when the strap is unwound.

FIG. 2D is an enlarged, partial top view of the strap of FIGS. 2A-2C.

FIGS. 2E and 2F depict cross-sectional views of the strap of FIGS. 2A-2C taken along line 2E-2E from FIG. 2D and line 2F-2F from FIG. 2B, respectively.

FIGS. 3 and 4 depict a first embodiment and second embodiment, respectively, of fasteners usable to affix some of the present flexible straps to the skin of a patient.

FIGS. 5A-5C depict insertion of the piercing bodies of fasteners through openings of the strap of FIGS. 2A-2C.

FIG. 6 depicts the separate sterilized containers for scissors and a writing instrument that can be part of various embodiments of the present kits.

FIG. 7 depicts a second embodiment of the present kits that includes a container that is sealed and contains two flexible straps and fasteners usable to secure the straps to the patient.

FIGS. 8A-8C depict a step of some of the present methods in which first and second fastening points are marked on the skin of the patient. In the embodiment shown, the markings are for a below-the-knee amputation procedure for a leg.

FIGS. 9A-9D depict a step of some of the present methods in which a flexible strap is fastened to a first part of the skin of the patient using fasteners that extend through first and second openings of the strap.

FIG. 10 depicts a step of some of the present methods in which an excess segment of the strap is cut after the strap has been fastened to the skin of the patient.

FIGS. 11A-11C depict a step of some of the present methods in which the first segment of the incision guide is marked along one of the lengthwise edges of the strap.

FIGS. 12A and 12B depict a step of some of the present methods in which a flexible strap is fastened to a second part of the skin of the patient after the first segment of the incision guide is marked.

FIGS. 13A-13D depict a step of some of the present methods in which the second segment of the incision guide is marked along one of the lengthwise edges of the strap.

FIGS. 14A and 14B depict a step of some of the present methods in which the skin is cut along the first and second segments of the incision guide, the part of the body circumscribed by the incision guide—as shown, a below-the-knee portion of a leg—is amputated, and the skin “flaps” of the first and second parts of the skin are joined.

FIGS. 15A-15E depict steps of some of the present methods that are substantially the same as those shown in FIGS. 8A-8C, 9A-9D, 10, 11A-11C, 12A and 12B, 13A-13D, and 14A and 14B, the primary exception being that the incision guide is marked for a mastectomy of a breast.

### DETAILED DESCRIPTION

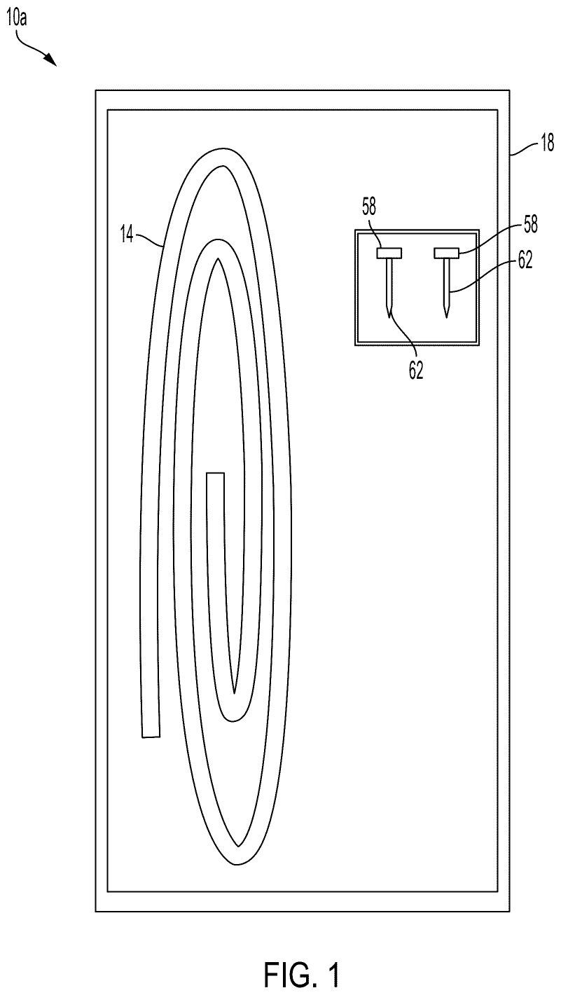

Referring now to the drawings, FIG. 1 depicts a first embodiment 10a of some of the present surgical stencil kits. Surgical stencil kit 10a can comprise: a flexible strap 14 and a container 18 that is sealed and contains the flexible strap for use in surgeries. Flexible strap 14 can be sterile, optionally such that a Sterility Assurance Level of the strap is less than or equal to any one of, or between any two of, 10⁻⁶, 0.9×10⁻⁶, 0.8×10⁻⁶, 0.7×10⁻⁶, 0.6×10⁻⁶, or 0.5×10⁻⁶. To maintain the sterility of flexible strap 14 before it is used, container 18 that contains strap 14 can be sealed and can optionally comply with ISO 11607-1:2019. The sterility of flexible strap 14 can help ensure that the strap can be safely used in surgical procedures like amputations, mitigating the risk of infection and potential surgical complications stemming therefrom.

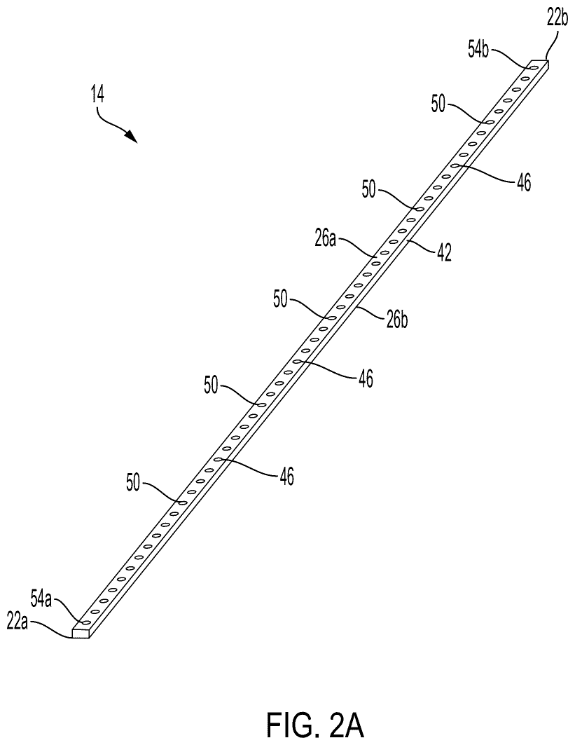

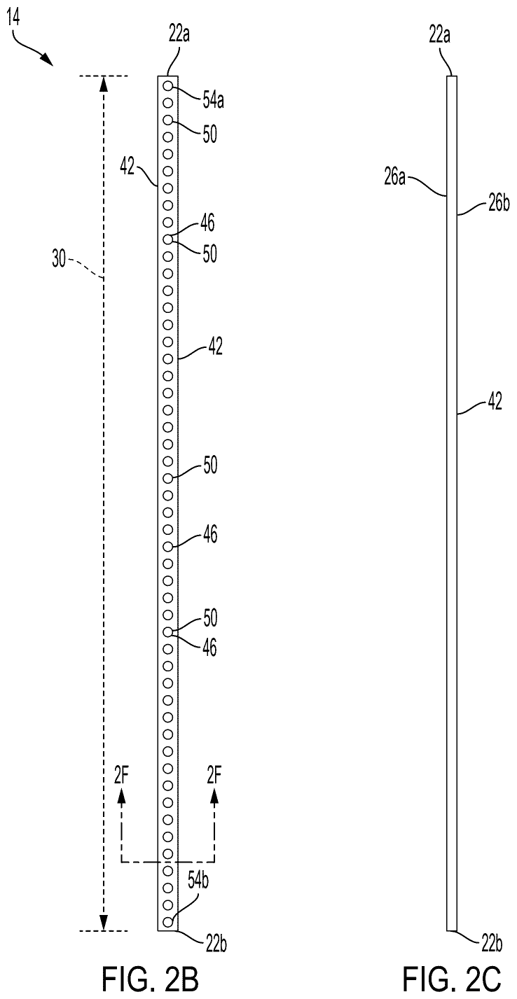

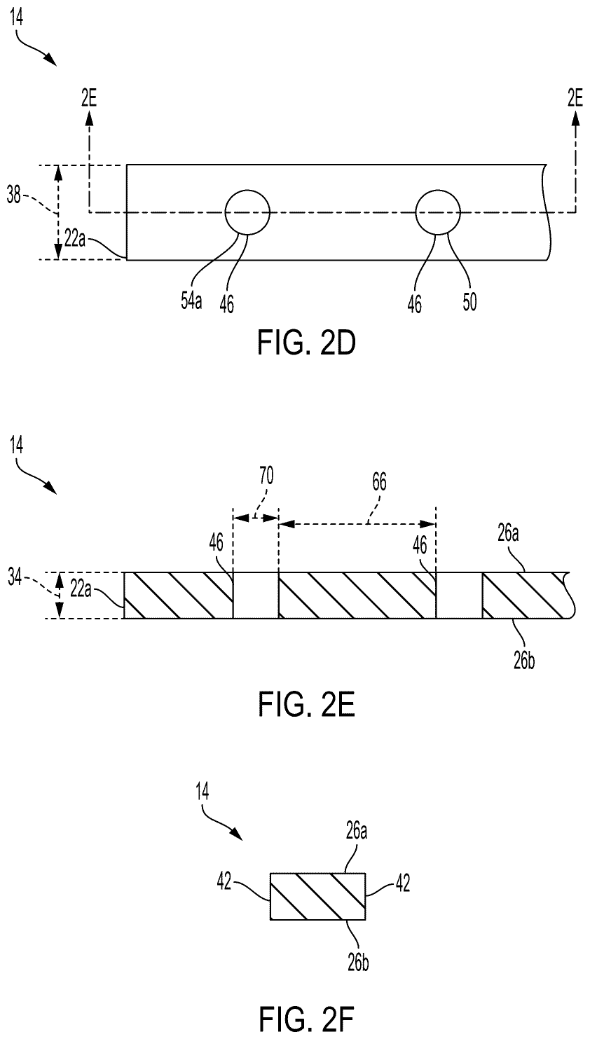

Now referring additionally to FIGS. 2A-2F, flexible strap 14 can extend between opposing first and second ends 22a and 22b, and can have opposing upper and lower surfaces 26a and 26b. Flexible strap 14 can have a size and shape that facilitates its use as a surgical stencil, as described in further detail below. A length 30, measured between first end 22a and second end 22b, of flexible strap 14 can be large compared to thickness 34—measured between the strap's upper and lower surfaces 26a and 26b—and width 38 of the strap to promote the strap's flexibility such that the strap can be deformed appropriately to fit around a body part of a patient, ensuring accurate marking of incision guides. For example, length 30 can be greater than or equal to any one of, or between any two of, 20, 25, 30, 40, 60, 70, 80, 90, 100, 110, 120, 130, 140, 150, 200, 300, or 400 times (e.g., at least 60 or 100 times) each of thickness 34 and width 38 of the strap. Furthermore, strap 14's length 30 can be sufficient to permit marking about various body parts, for example, greater than or equal to any one of, or between any two of, 20, 30, 40, 60, 80, or 100 centimeters (e.g., between 20 and 100 centimeters or at least 60 centimeters). To further facilitate the flexibility of strap 14 and promote the strap's ability be applied precisely to mark a segment of an incision guide, width 38 of the strap can be relatively small, such as less than or equal to any one of, or between any two of, 1, 2, 3, 5, 8, or 10 millimeters (e.g., between 1 and 10 millimeters or between 3 and 7 millimeters, such as about 5 millimeters). Strap 14 can additionally have a thickness and shape to facilitate the marking of a segment of an incision guide along one of its lengthwise edges 42, which with an appropriate thickness and shape can better support a marking utensil than conventional apparatuses. For example, thickness 34 of flexible strap 14 can be less than or equal to any one of, or between any two of, 1, 2, 3, 5, 8, or 10 millimeters (e.g., between 1 and 10 millimeters or between 1 and 3 millimeters, such as about 2 millimeters). Additionally, as shown in FIG. 2F, a cross-section of flexible strap 14 that includes a plane that is perpendicular to length 30 of the strap can be rectangular to promote the ability of lengthwise edges 42 to support a marking utensil.

Flexible strap 14 can comprise a material that allows it to conform about a body part of a patient and that can resist deformation when supporting a marking utensil, such as a polymeric material and/or a metal. For example, strap 14 can comprise a polymeric material having a modulus of elasticity that can be greater than or equal to any one of, or between any two of, 1.25, 1.75, 2.5, 3.5, or 4 gigapascals (e.g., between 1.25 and 4 gigapascals), such as nylon. If strap 14 comprises a metal, the metal can be coated with a polymeric material such as silicone (e.g., such that the polymeric material defines upper surface 26a and/or lower surface 26b of the strap) such that the polymeric material mitigates slippage on a patient's skin during use.

Strap 14 can comprise a plurality of openings 46 that can each extend through the strap from upper surface 26a to lower surface lower surface 26b. Each of openings 46 can be sized such that, as described in further detail below, a piercing body 62 of a fastener 58 can extend through the opening and pierce skin of a patient to secure strap 14 to the patient when marking a segment of an incision guide.

Openings 46 can include a plurality of intermediate openings 50 that are each disposed 1) closer to first end 22a of the strap than is at least one of the other openings, and 2) closer to second end 22b of the flexible strap than is at least one of the other openings. Openings 46 can also include first and second end openings 54a and 54b, the first end opening disposed closer to first end 22a than are each other of the openings and the second end opening disposed closer to second end 22b than are each other of the openings. This arrangement of openings 46 can allow the openings to serve as a reference for the length of a segment of an incision guide drawn using strap 14, which can facilitate an incremental length selection when utilizing the strap to mark a segment of an incision guide and the marking of multiple segments of an incision guide that are of substantially the same length. In particular, with openings 46 having different positions along strap 14's length 30, a length of a segment of an incision guide marked using the strap can be selected and referenced by 1) selecting two of the openings through which piercing bodies 62 of fasteners 58 can be inserted to secure the strap to a patient and 2) marking the segment of the incision guide along a portion of one of lengthwise edges 42 that is disposed between the selected openings. Compared to having only one opening 46 at each end 22a and 22b of strap 14, the multitude of openings affords a user flexibility in which portion the strap is used to mark a segment of an incision guide having a desired length while also allowing the user to keep track of the length of the segment of the incision guide marked based on which of the openings are used for fastening, which can facilitate a more precise marking of multiple segments of the incision guide. To promote precision in the selection of a referenceable length for the portion of strap 14 used to mark a segment of an incision guide, openings 46 can be arranged such that the distance between each opening is relatively small. For example, for each opening 46 of flexible strap 14, a distance 66 between the opening and at least one—optionally at least two—other of the openings can be less than or equal to any one of, or between any two of, 0.2, 0.3, 0.4, 0.5, 0.6, 0.7, 0.8, 0.9, 1, 1.5, 2, 2.5, or 3 centimeters (e.g., less than or equal to 3 centimeters). To further promote this incremental precision, strap 14 can have a large number of openings 46, such as greater than or equal to any one of, or between any two of, 8, 9, 10, 11, 12, 13, 14, 15, 16, 17, 18, 19, 20, 21, 22, 23, 24, 25, 26, 27, 28, 29, or 30 openings (e.g., at least 15 openings), optionally wherein the number of intermediate openings 50 of the strap is greater than or equal to any one of, or between any two of, 10, 11, 12, 13, 14, 15, 16, 17, 18, 19, 20, 21, 22, 23, 24, 25, 26, 27, or 28 openings (e.g., at least 13 intermediate openings).

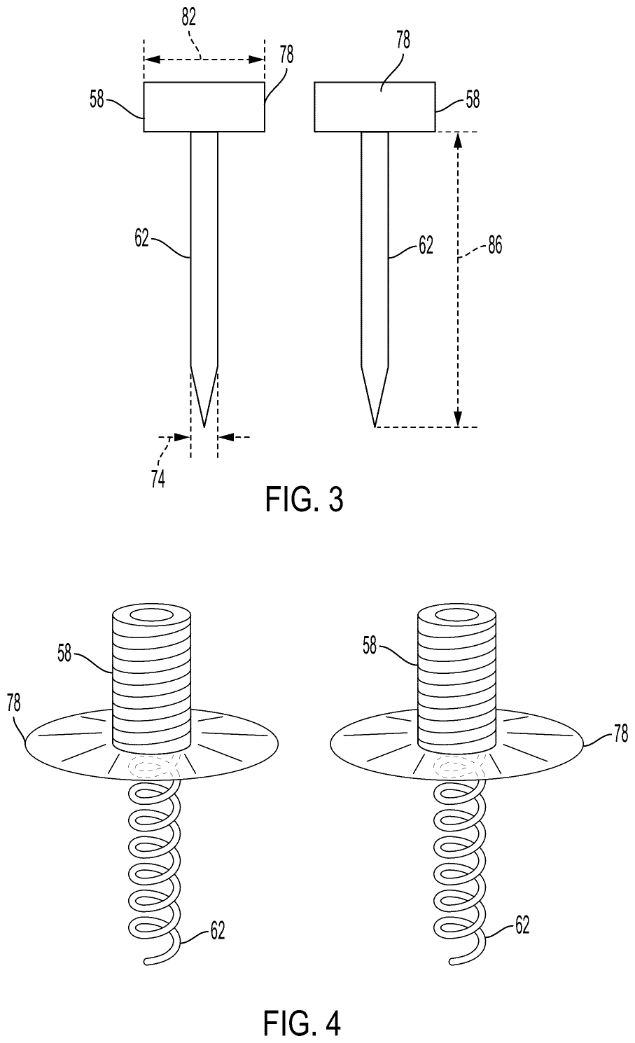

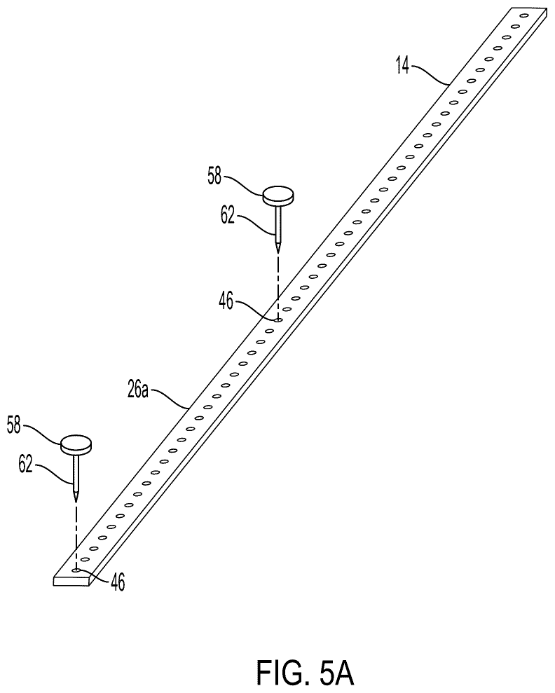

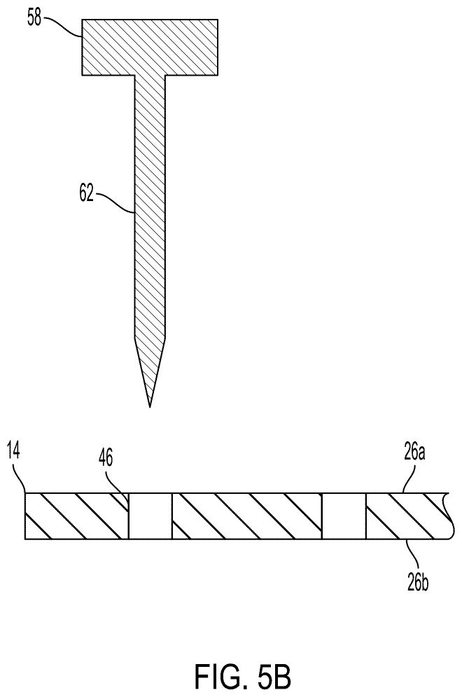

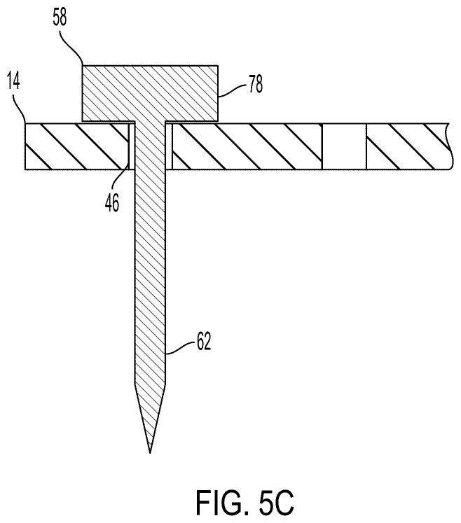

As noted above, each of openings 46 extending through strap 14 can be designed to accommodate a piercing body 62 of a fastener 58, wherein the fastener is designed to fasten the strap to the skin of a patient. For example, each of the openings 46 of flexible strap 14 can have a maximum transverse dimension 70 (e.g., diameter), measured in a direction that is perpendicular to the thickness 34 of the strap, that is greater than or equal to any one of, or between any two of, 1, 1.5, 2, 2.5, 3, 3.5, 4, 4.5, 5, 5.5, or 6 millimeters (e.g., between 1 and 6 millimeters). In the embodiment shown, kit 10a can comprise a plurality of fasteners 58 that each include a piercing body 62 sized to extend through one of the openings 46 of flexible strap 14 and pierce the skin of a patient, although in other embodiments the kit need not include fasteners (e.g., if the fasteners are supplied by an end user). Referring additionally to FIGS. 3 and 5A-5C, a maximum transverse dimension 74 (e.g., diameter) of piercing body 62 of each fastener 58 can be less than that of each of the openings 46 so that the piercing body can fit through any of the openings of strap 14. For example, maximum transverse dimension 74 of piercing body 62 can be less than or equal to any one of, or between any two of, 6, 5.5, 5, 4.5, 4, 3.5, 3, 2.5, 2, 1.5, or 1 millimeters (e.g., between 1 and 5 millimeters). Fasteners 58 can also each have a head 78 that can have a maximum transverse dimension (e.g., diameter) 82 that can be bigger than the maximum transverse dimension 70 of each opening 46 such that when the piercing body 62 of the fastener extends through one of the openings and into the skin of a patient, the fastener head can engage one of opposing surfaces 26a and 26b to secure the strap to the skin of the patient. Length 86 of piercing body 62 of each of fasteners 58 can be larger than thickness 34 of strap 14 such that the piercing body can extend through and beyond one of openings 46 and achieve adequate engagement with the skin of a patient, such as a length that is greater than or equal to any one of, or between any two of, 5, 6, 7, 8, 9, 10, 11, 12, 13, 14, or 15 millimeters (e.g., between 8 and 12 millimeters).

Fasteners 58 can be of any type suitable to secure strap 14 to the skin of a patient. For example, as shown in FIG. 3, fasteners 58 can each comprise a fastener such as a pin or tack having a straight piercing body 62 that can be pressed into the skin of a patient. However, referring to FIG. 4, in other embodiments each of fasteners 58 can have a helical piercing body 62 that can be screwed into the skin of a patient. Each of fasteners 58 can be sterile (e.g., with a Sterility Assurance Level that is less than or equal to any one of, or between any two of, 10⁻⁶, 0.9×10⁻⁶, 0.8×10⁻⁶, 0.7×10⁻⁶, 0.6×10⁻⁶, or 0.5×10⁻⁶) such that they can be safely used in surgical procedures, mitigating the risk of infection. As shown in FIG. 1, fasteners 58 can be contained within container 18.

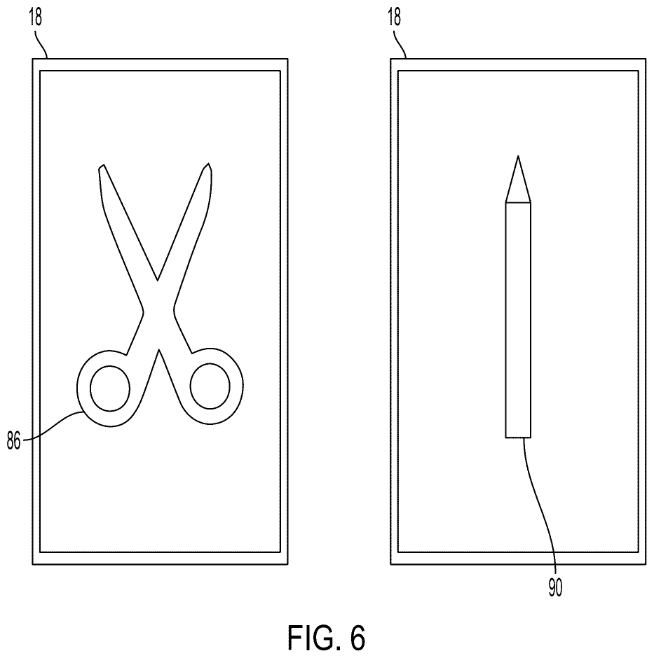

Referring to FIG. 6, some kits can also comprise a cutting instrument 86 (e.g., scissors) and/or a marking utensil 90 (e.g., a skin pen). Cutting instrument 86 can be used to, for example, cut strap 14 during use, and marking utensil 90 can be used to mark an incision guide. Cutting instrument 86 and marking utensil 90 can each be sterile (e.g., having any of the above-described Sterility Assurance Levels) and can be contained in a sealed container 18—either the same container that contains strap 14 or a different container—to maintain sterility before use.

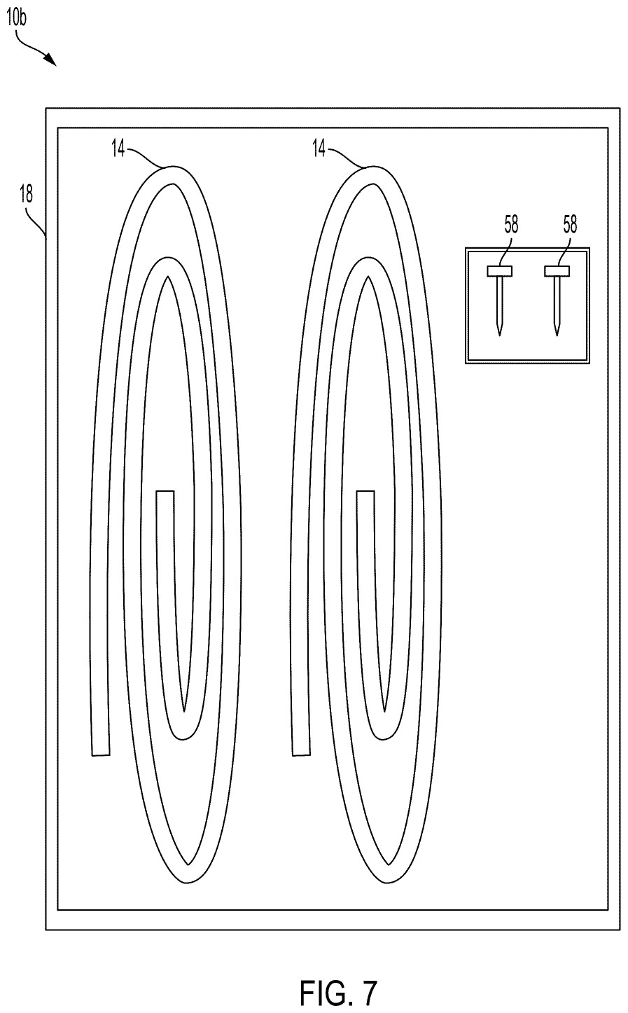

Referring to FIG. 7, shown is a second kit 10b that is substantially the same as kit 10a, the primary exception being that kit 10b includes two flexible straps 14 instead of a single strap. As described in further detail below, two straps 14 can be used to either concurrently or consecutively mark two different segments of an incision guides

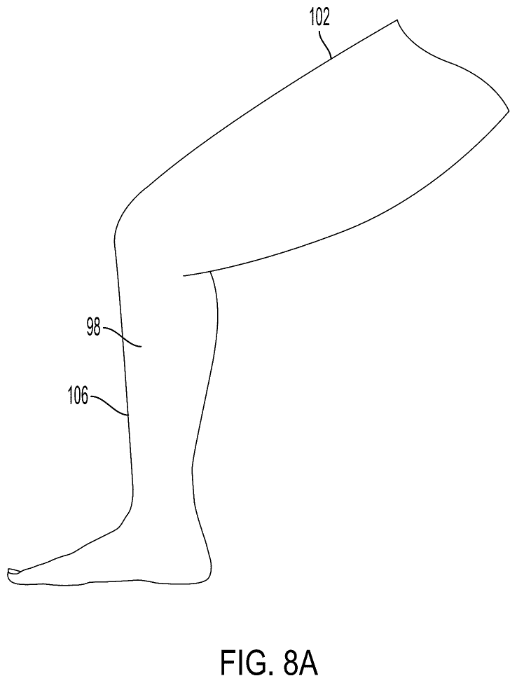

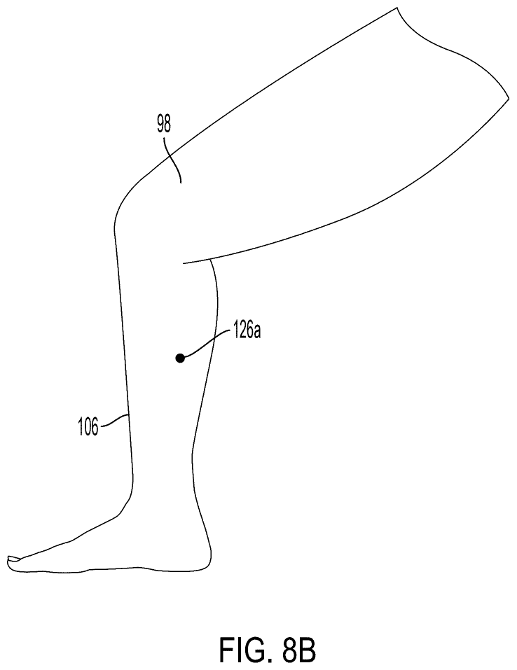

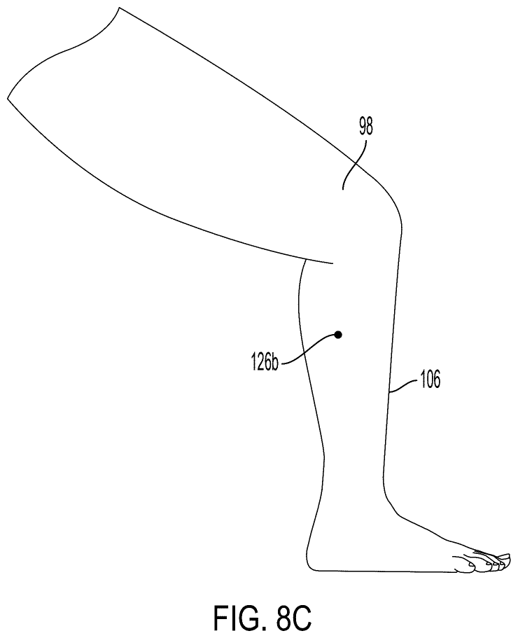

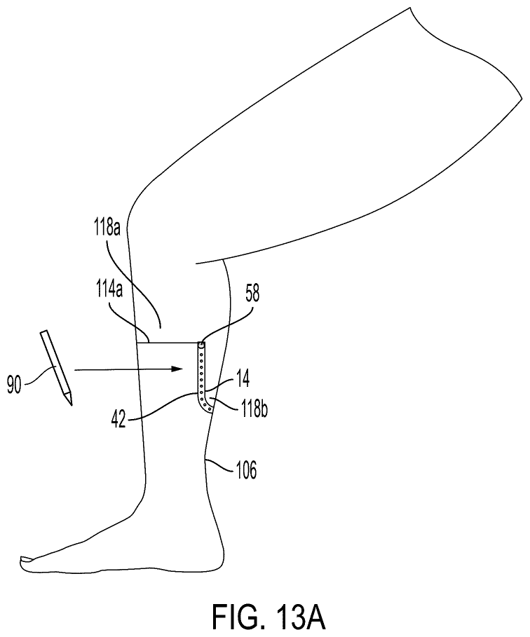

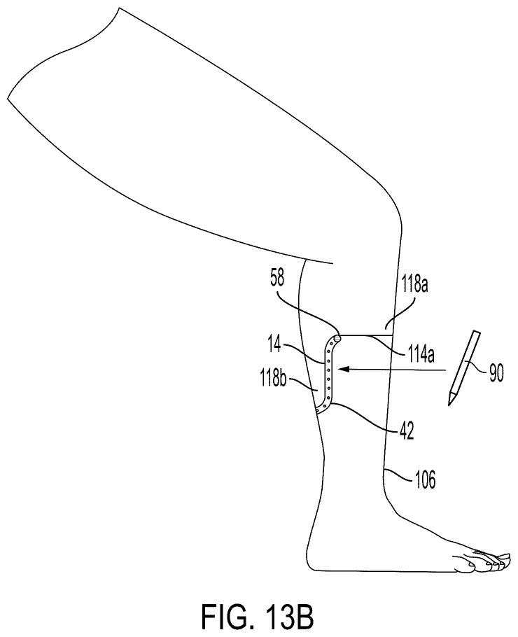

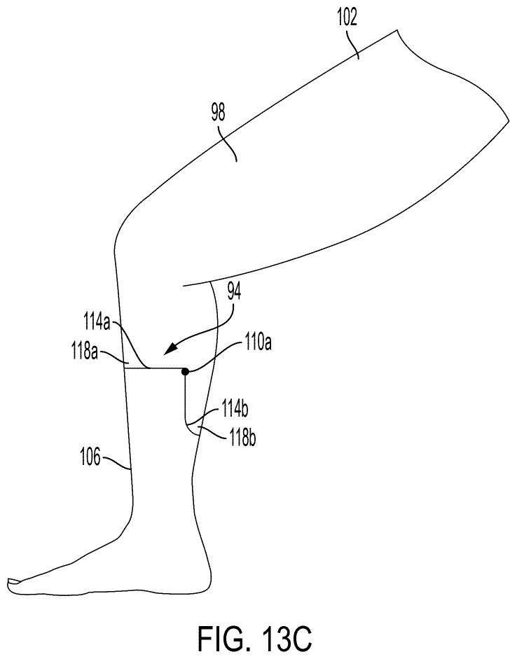

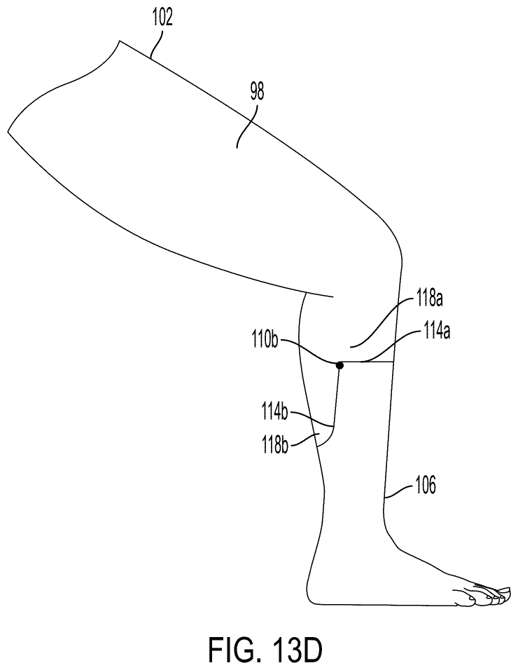

Referring now to FIGS. 8A-14B, illustrated are some of the present methods for performing an amputation on a patient. As shown, some methods comprise marking an incision guide (e.g., 94) on the skin (e.g., 98) of a patient (e.g., 102) such that, as shown in FIGS. 13C and 13D, the incision guide circumscribes a part (e.g., 106) of the body of the patient. The marked incision guide can include 1) a first vertex (e.g., 110a) and a second vertex (e.g., 110b), 2) a first segment (e.g., 114a) that extends along a first part (e.g., 118a) of the skin of the patient between the first vertex and second vertex, and 3) a second segment (e.g., 114b) that extends along a second part (e.g., 118b) of the skin of the patient between the first vertex and the second vertex. As described in further detail below, the second segment can have a length, measured between the first and second vertices, that is substantially the same as the length of the first segment, measured between the first and second vertices, and the segments can be marked in a way that facilitates the achievement of this length similarity. This ensures that the opposing flaps (e.g., 122a and 122b) of skin cut along the segments of the incision guide can have substantially the same length such that they can be easily conjoined to finish the procedure.

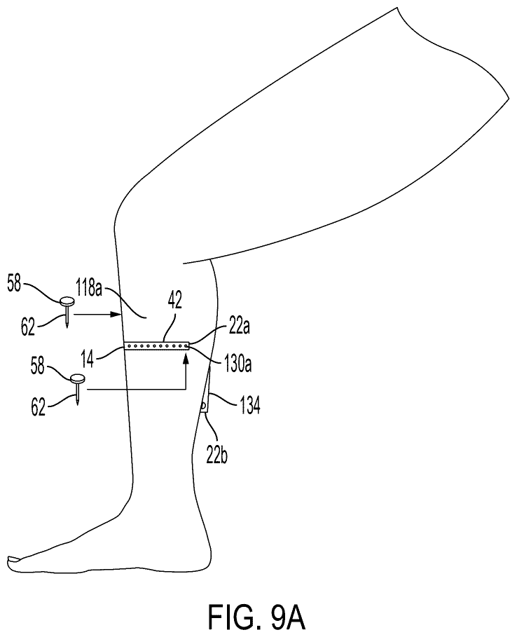

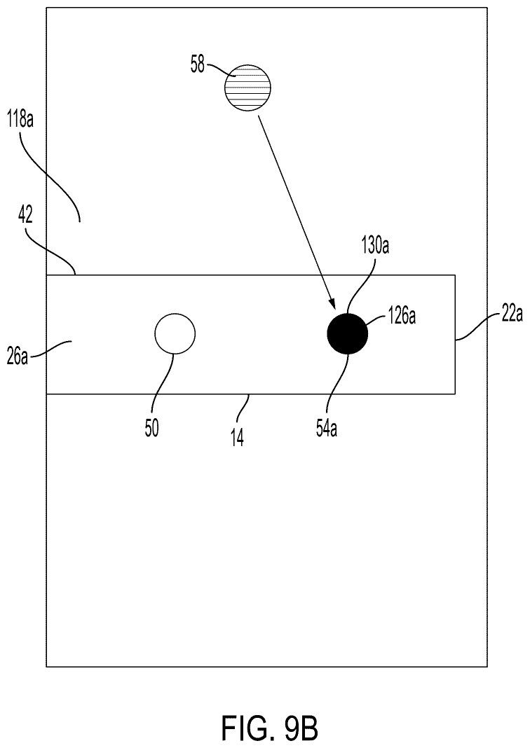

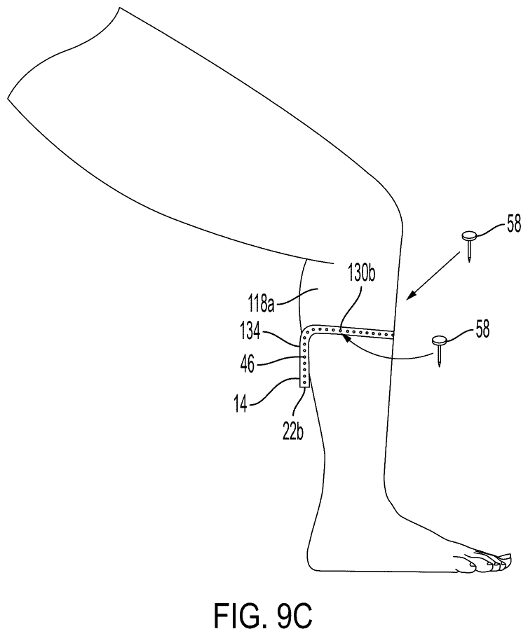

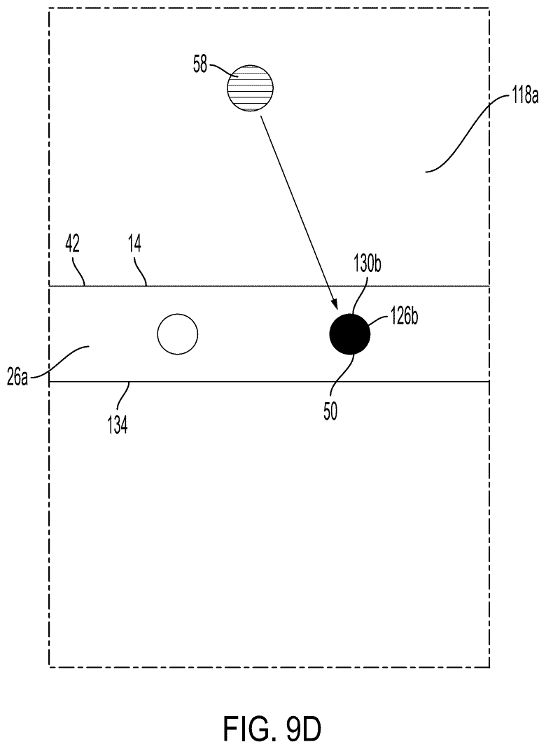

Marking the incision guide can be performed at least in part with a flexible strap (e.g., 14), which can be any of those described above that extends between opposing first and second ends (e.g., 22a and 22b) and has a plurality of openings (e.g., 46) that each extend through the strap from an upper surface (e.g., 26a) of the strap to an opposing lower surface (e.g., 26b) of the strap. Referring specifically to FIGS. 8A-8C, to facilitate the use of the flexible strap(s), before marking the incision guide, first and second fastening points (e.g., 126a and 126b) can be marked on the skin of the patient to indicate where on the patient to fasten the strap. Referring next to FIGS. 9A-9D, the strap can then be disposed on the first part of the skin of the patient and positioned in a manner satisfactory to the surgeon for marking of the first segment of the incision guide. If fastening points were marked, a first one of the openings (e.g., 130a) of the strap can overlie the first fastening point and a second one of the openings (e.g., 130b) of the strap that is disposed closer to the second end of the strap than is the first opening can overlie the second fastening point. When the strap is placed appropriately to delineate where the first segment of the incision guide will be marked, the strap can then be fastened to the first part of the skin of the patient. The flexible strap can be fastened to the first part of the skin of the patient at least by, for each of the first and second openings, inserting a piercing body (e.g., 62) of a fastener (e.g., 58) through the opening and into the skin of the patient. As explained above, which of the openings of the strap to use as the first and second openings through which the strap is fastened can be chosen based at least in part on the length of the first segment of the incision guide to be marked (e.g., with the portion of the strap disposed between the first and second openings being used to mark the segment of the incision guide). For example, the first and second openings can be ones that, when the strap is fastened to the first part of the skin, are positioned close to where the first and second vertices, respectively, of the incision guide will be marked, optionally such that a minimum distance (e.g., a straight-line distance through the body part) between the first opening and the second opening of the strap when fastened to the first part of the skin of the patient is substantially the same as a minimum distance (e.g., a straight-line distance through the body part) between the first vertex and second vertex of the incision guide when the incision guide is marked. In the embodiment shown, the first opening is one of the end openings (e.g., 54a and 54b) of the strap and the second opening is one of the intermediate openings (e.g., 50) of the strap, although in other embodiments (e.g., depending on the length of the segment to be drawn), the first opening can be one of the intermediate openings and/or the second opening can be one of the end openings.

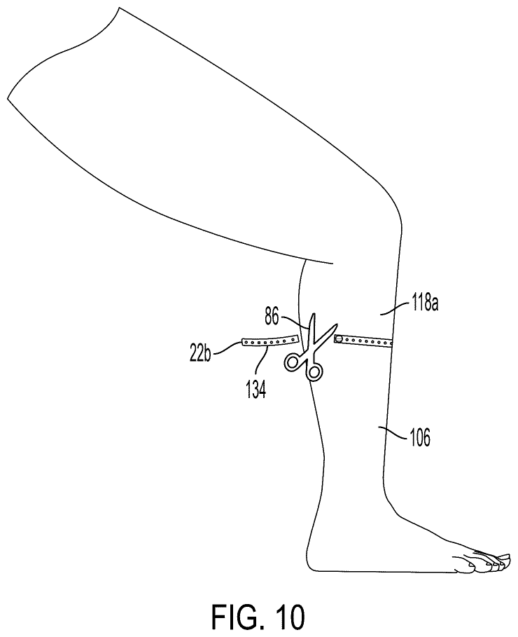

In some methods, a length of the first segment of the incision guide to be marked is less than a length (e.g., 30) of the strap such that a segment (e.g., 134) of the strap that includes the first end or the second end of the strap and is not fastened to the first part of the skin of the patient (e.g., does not include the first and second openings of the strap) will not be used to mark the first segment of the incision guide. As shown in FIG. 10, in such methods, after fastening the flexible strap to the first part of the body of the patient with fasteners, that excess segment of the strap can be removed by cutting the strap, such as with a cutting instrument like scissors. Doing so can facilitate handling of the strap, although in some methods the excess segment need not be removed.

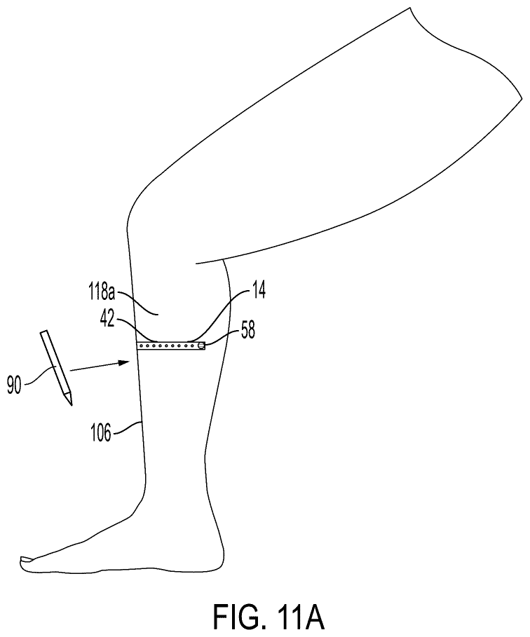

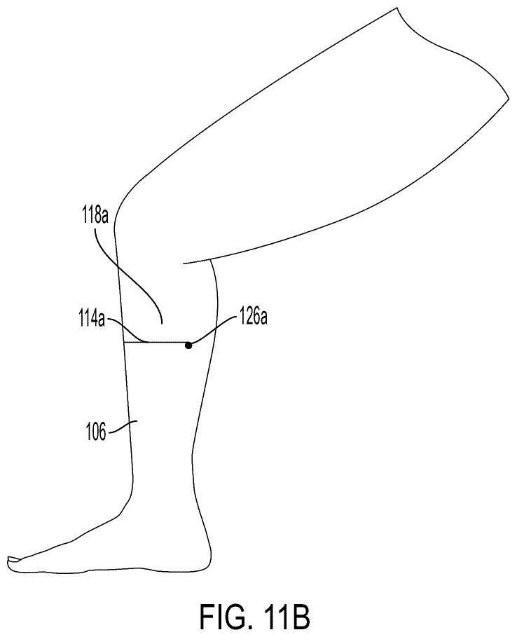

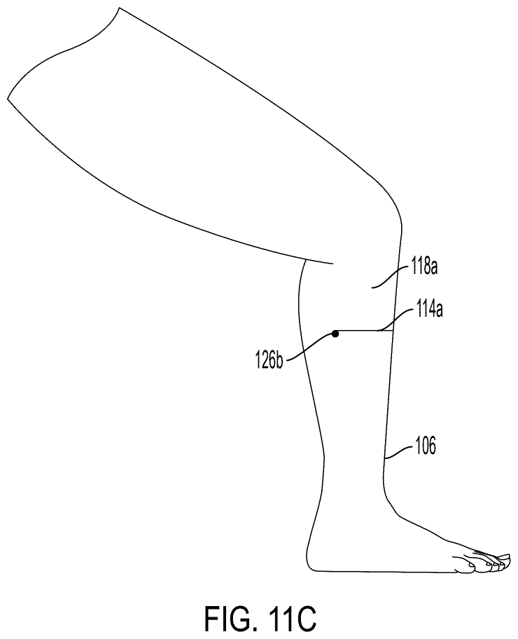

Referring to FIGS. 11A-11C, while the strap is fastened to the first part of the skin of the patient, the first segment of the incision guide can be marked, using a writing utensil (e.g., 90), for example, a skin pen. The incision guide can be marked along one of the opposing lengthwise edges (e.g., 42) of the strap that each extend between the upper surface and the lower surface of the strap, which can support the writing utensil as the writing utensil is drawn along the lengthwise edge to mark the first segment of the incision guide. After marking the first segment of the incision guide, the fasteners and flexible strap can then be removed from the first part of the skin of the patient (FIGS. 11B and 11C).

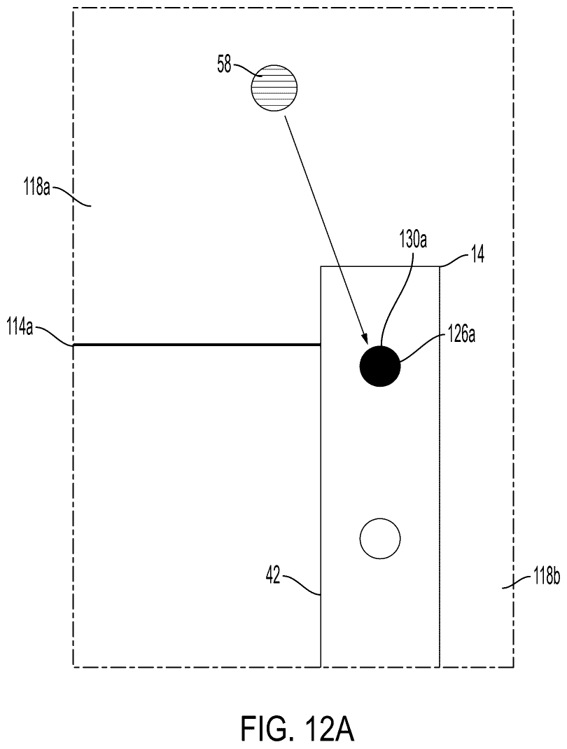

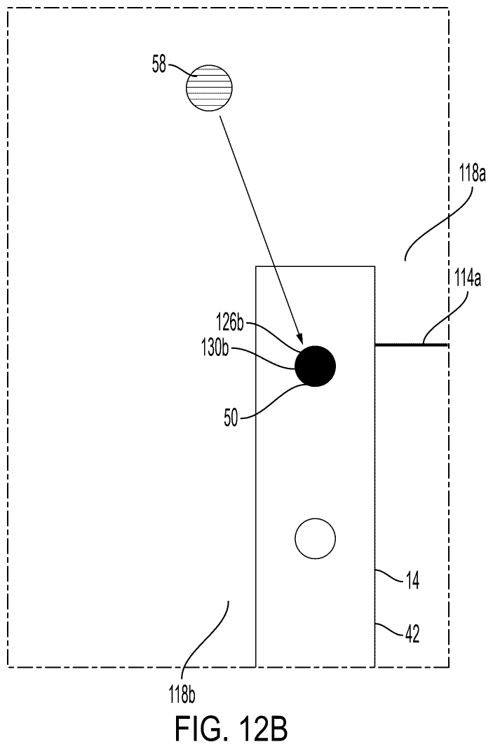

In some methods, the same strap used to mark the first segment of the incision guide can be used to mark the second segment of the incision guide. To do so, the strap—after being removed from the first part of the skin of the patient—can be disposed on the second part of the skin of the patient such that one of its lengthwise edges is disposed along where the second segment of the incision guide is to be drawn. If fastening points were marked, the strap can be positioned such that the first opening of the strap overlies one of the first and second fastening points and the second opening of the strap overlies the other of the first and second fastening points, which can facilitate appropriate positioning of the strap. Then, as is shown in FIGS. 12A and 12B, the strap can be fastened to the second part of the skin of the patient at least by, for each of the first opening and second opening of the strap, inserting a piercing body of a fastener through the opening and into the skin of the patient. The fasteners used can be the same as those used when securing the strap to mark the first segment of the incision guide, or can be new fasteners. While the strap is fastened to the second part of the skin of the patient, the second segment of the incision guide can be marked along one of the lengthwise edges of the strap using the writing utensil, as is shown in FIGS. 13A-13D. The fasteners and strap can then be removed, which can leave a complete incision guide having the first and second segments connected at the first and second vertices as shown in FIGS. 13C and 13D. By following the aforementioned procedure where the same first and second openings of the strap are used to secure the strap both when marking the first segment and the second segment (e.g., with the same portion of the strap between the first and second openings being used when marking the first and second segments), the user can readily employ the strap to ensure that the first and second segments of the incision guide have substantially the same length.

In other methods, the first segment of the incision guide can be marked through the use of the strap as described above after the second segment of the incision guide is marked through a conventional technique (e.g., free-hand drawing). In such embodiments, the user (e.g., a surgeon) can mark the second segment without the use of the strap as a guide. Then, the strap can be used to measure the length of the second segment with reference to the openings of the strap. The length of the second segment of the incision guide can delineate which of the openings of the strap to use for the first and second openings for fastening the strap to the skin of the patient to mark the first segment of the incision guide.

In yet other methods, two straps can be used to mark the first and second segments of the incision guide on the skin of the patient. In these methods, the first strap can be used to mark the first segment and the second strap can be used to mark the second segment, wherein the straps can be fastened to the skin of the patient at the same time or at different times. If the straps are employed and fastened to the skin of the patient at the same time, a single set of fasteners can be utilized, fastening the straps to the patient at the same respective first and second openings on each strap. The lengthwise separation between the first and second openings of the first strap can be substantially the same as the lengthwise separation between the first and second openings of the second strap, thereby ensuring that the distance along each strap's lengthwise edges (and thereby the marked segments of the incision guide) will be of substantially the same length even though two straps are utilized.

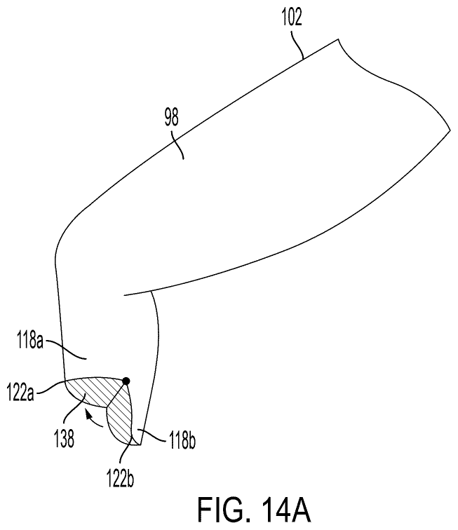

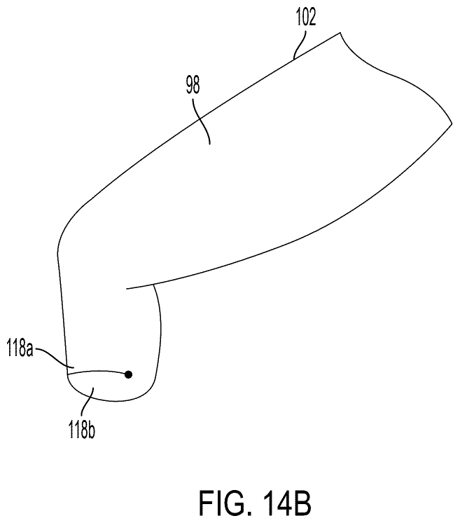

After the incision guides have been marked, the skin of the patient can be cut along the incision guide and the part of the body can be amputated such that the patient has an open wound (e.g., 138) as shown in FIG. 14A. The first part of the skin can then be coupled to the second part of the skin such that the first and second parts of the skin overlie the open wound as shown in FIG. 14B. As each segment of the incision guide was marked to have substantially the same length—a result facilitates by utilizing the one or more straps—the cuts along each segment of the incision guide can yield flaps of skin that have substantially the same length and will thus fit together such that the first and second parts of the skin can cover the open wound with little if any excess (or shortage) of skin present. This reduces the likelihood of error during a procedure and reduces the risk that a patient encounters wound complications after the opposing flaps of skin are joined together.

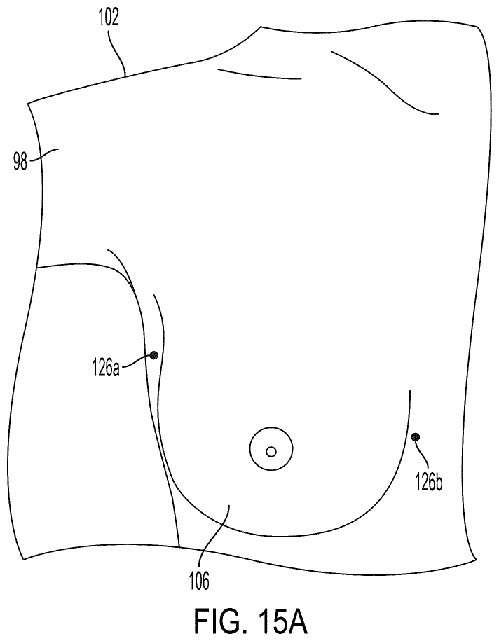

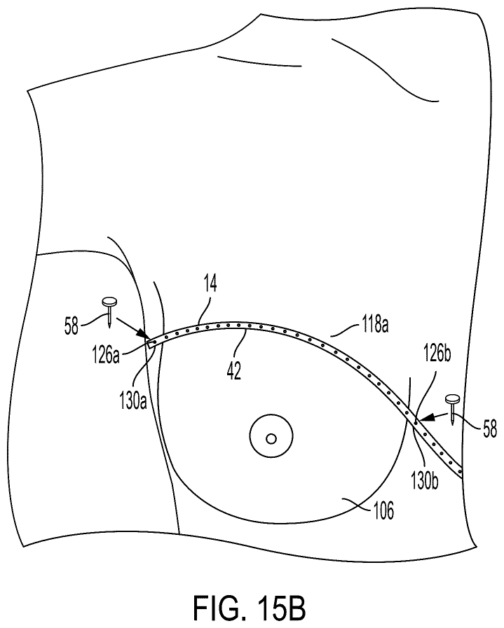

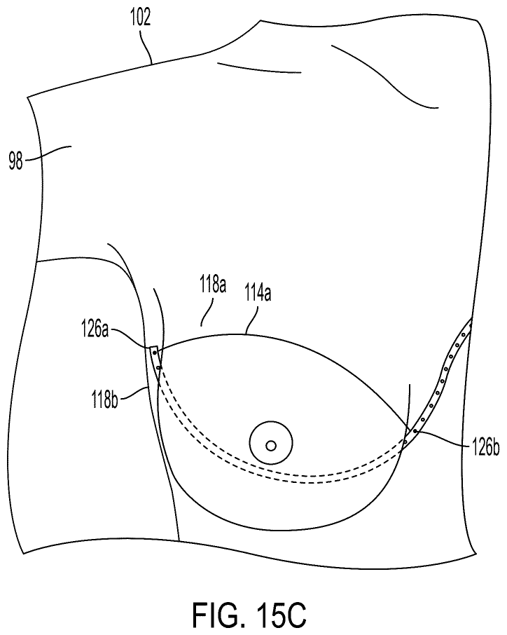

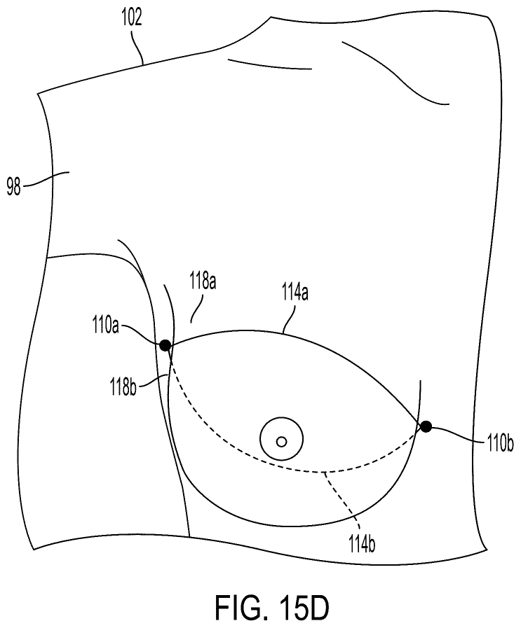

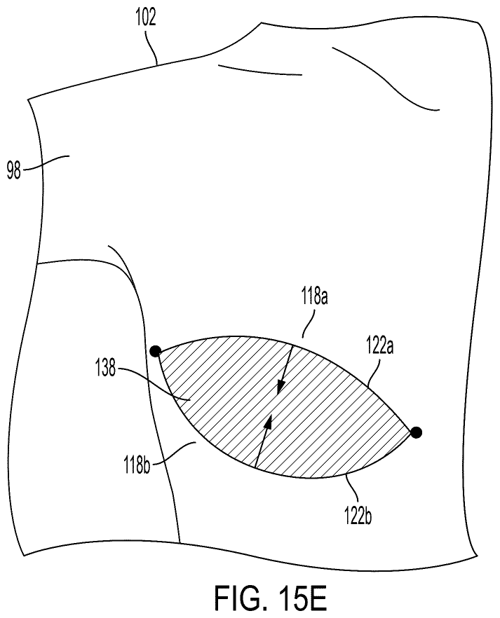

The methodologies described above can be applied to a wide variety of surgeries and/or amputations, including appendage (e.g., leg, arm, hand, foot, and/or the like) amputations, mastectomies, abdominoplasties, and/or the like. In the embodiment shown, the incision guide is drawn for a leg amputation. To further illustrate, FIGS. 15A-15E shows the same methodology as shown above for a leg amputation applied for a mastectomy procedure of a breast, where the first fastening point and the second fastening point can be marked on the skin of the patient (FIG. 15A), the strap can be disposed on the first part of the skin and fixed thereto with the aforementioned fasteners once in an appropriate position to mark the first segment of the incision guide (FIG. 15B), and the strap can be disposed on and fastened to the second part of the skin after the first segment is marked to mark the second segment of the incision guide (FIG. 15C), which can yield an incision guide having first and second segments that have substantially the same length and fully circumscribe the breast (FIG. 15D). Finally, as shown in FIG. 15E, incisions can be made along the incision guide and the breast can be removed. As the segments of the incision guide had substantially the same length, the resulting cuts of skin can also have lengths that are substantially the same. Thus, there can be a reduced risk of complications both during and after the surgical procedure due to misaligned cuts of skin.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the products, systems, and methods are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A kit comprising:
a flexible strap that extends between opposing first and second ends and has:
opposing upper and lower surfaces;
a plurality of openings that each extend through the strap from the upper surface to the lower surface, wherein the openings include a plurality of intermediate openings that are each disposed:
closer to the first end of the strap than is at least one other of the openings; and
closer to the second end of the strap than is at least one other of the openings; and
a length, measured between the first and second ends, that is at least 20 times each of a thickness and a width of the strap, the thickness measured between the upper and lower surfaces;
wherein the strap is sterile; and
a container that is sealed and contains the strap.

2. The kit of claim 1, comprising:
a plurality of fasteners that each include a piercing body sized to extend through one of the openings of the flexible strap and configured to pierce skin of a patient;
wherein each of the fasteners is sterile.

3. The kit of claim 1, wherein for each of the openings of the strap, a distance between the opening and at least one other of the openings is less than or equal to 3 centimeters.

4. The kit of claim 1, wherein:
the length of the strap is between 20 and 100 centimeters;
the thickness of the strap is between 1 and 10 millimeters; and
the width of the strap is between 1 and 10 millimeters.

5. The kit of claim 1, wherein the flexible strap comprises a polymeric material having a modulus of elasticity that is between 1.25 and 4 gigapascals.

6. The kit of claim 1, wherein each of the openings of the strap has a maximum transverse dimension, measured in a direction that is perpendicular to the thickness of the strap, that is between 1 and 6 millimeters.

7. The kit of claim 1, wherein the intermediate openings comprise ten or more intermediate openings.

8. The kit of claim 1, wherein a cross-section of the strap that includes a plane that is perpendicular to the length of the strap is rectangular.

9. The kit of claim 1, wherein a Sterility Assurance Level of the strap is less than or equal to 10⁻⁶.

10. The kit of claim 1, wherein the container complies with ISO 11607-1:2019.

11. A method for performing an amputation on a patient, the method comprising marking an incision guide on skin of the patient, wherein:
the incision guide circumscribes a part of a body of the patient and includes:
first and second vertices;
a first segment that extends along a first part of the skin of the patient between the first and second vertices; and
a second segment that extends along a second part of the skin of the patient between the first and second vertices and has a length, measured between the first and second vertices, that is substantially the same as a length, measured between the first and second vertices, of the first segment; and
marking the incision guide comprises:
disposing a flexible strap on the first part of the skin, the strap extending between opposing first and second ends and having a plurality of openings that each extend through the strap from an upper surface of the strap to an opposing lower surface of the strap;
fastening the strap to the first part of the skin of the patient at least by, for each of a first one of the openings of the strap and a second one of the openings of the strap that is disposed closer to the second end of the strap than is the first opening, inserting a piercing body of a fastener through the opening and into the skin of the patient; and
while the strap is fastened to the first part of the skin of the patient, marking the first segment of the incision guide along one of opposing lengthwise edges of the strap that each extend between the upper and lower surfaces of the strap.

12. The method of claim 11, wherein marking the incision guide comprises, after marking the first segment of the incision guide:
removing the strap from the first part of the skin of the patient; and
after removing the strap from the first part of the skin of the patient:
disposing the strap on the second part of the skin of the patient;
fastening the strap to the second part of the skin of the patient at least by, for each of the first and second openings of the strap, inserting a piercing body of a fastener through the opening and into the skin of the patient; and
while the strap is fastened to the second part of the skin of the patient, marking the second segment of the incision guide along one of the lengthwise edges of the strap.

13. The method of claim 12, comprising:
before marking the incision guide, marking first and second fastening points on the skin of the patient;
wherein:
the first opening of the strap overlies the first fastening point and the second opening of the strap overlies the second fastening point when the strap is fastened to the first part of the skin of the patient; and
the first opening of the strap overlies one of the first and second fastening points and the second opening of the strap overlies the other of the first and second fastening points when the strap is fastened to the second part of the skin of the patient.

14. The method of claim 11, wherein:
the openings of the strap include a plurality of intermediate openings that are each disposed:
closer to the first end of the strap than is at least one other of the openings; and
closer to the second end of the strap than is at least one other of the openings; and
the first opening of the strap is one of the intermediate openings of the strap and/or the second opening of the strap is one of the intermediate openings of the strap.

15. The method of claim 14, wherein the intermediate openings of the strap comprise ten or more intermediate openings.

16. The method of claim 14, comprising, after fastening the strap to the first part of the body of the patient, cutting the strap to remove a segment of the strap that:
includes the first end of the strap or the second end of the strap; and
does not include the first and second openings of the strap.

17. The method of claim 11, comprising:
cutting the skin of the patient along the incision guide;
amputating the part of the body such that the patient has an open wound; and
coupling the first part of the skin to the second part of the skin such that the first and second parts of the skin overlie the open wound.

18. The method of claim 11, wherein a minimum distance between the first and second openings of the strap when the strap is fastened to the first part of the skin of the patient is substantially the same as a minimum distance between the first and second vertices of the incision guide.

19. The method of claim 11, wherein for each of the openings of the strap, a distance between the opening and at least one other of the openings is less than or equal to 3 centimeters.

20. The method of claim 11, wherein a cross-section of the strap that includes a plane that is perpendicular to a length of the strap, measured between the first and second ends of the strap, is rectangular.
